(19) United States
(12) Patent Application Publication
(10) Pub. No.: US 2002/0009509 A1
(43) Pub. Date: Jan. 24, 2002

(54) Soya extract containing lipids, process for its production, and pharmaceutical and cosmetic composition

(76) Inventor: Ezio Bombardelli, Milano (IT)

Correspondence Address: PENNIE & EDMONDS LLP, 1667 K STREET NW, SUITE 1000, WASHINGTON, DC, 20006

(21) Appl. No.: 09/917,905

(22) Filed: Jul. 31, 2001

Related U.S. Application Data
(63) Continuation of application No. PCT/EP00/00826, filed on Feb. 2, 2000.

(30) Foreign Application Priority Data
Feb. 3, 1999 (DE) 199 04352.3

(51) Int. Cl. A61K 35/78; A61K 31/685; A61K 31/7024

(52) U.S. Cl. 424/757; 514/23; 514/78

(57) ABSTRACT

A soya extract containing lipids, a process for its production, and a pharmaceutical and cosmetic composition containing said extract, which is intended in particular for the prevention or treatment of dry skin.

### CROSS REFERENCE TO RELATED APPLICATIONS

This application is a continuation of the U.S. national phase designation of PCT application no. PCT/EP00/00826, filed Feb. 2, 2000, the entire contents of which are incorporated herein by reference thereto.

### FIELD OF THE INVENTION

The present invention relates to novel extracts obtained by extraction of ripe whole soya beans or from oil-free soya flour, to their production and to formulations containing these extracts.

### BACKGROUND OF THE INVENTION

In JP 06228171-A, a purification of sphingomyelin by contacting soybean with a non-polar carrier and extracting sphingomyelin from non-polar carrier with a polar solvent is described.

### SUMMARY OF THE INVENTION

The invention relates to novel extracts obtained by extraction of ripe whole soya beans or from oil-free soya flour (Glycine max (L.) MERRIL, Leguminosae family), to their production and to formulations containing these extracts. The novel extracts are characterized by their content of sphingomyelins and phospholipids in defined ratios. In this context, “sphingomyelins” are taken to mean phospholipids derived from sphingosine and “phospholipids” are taken to mean phospholipids derived from glycerol.

It has been found that these extracts have significant effectiveness especially in the cosmetics field for the treatment of dry skin.

### DESCRIPTION OF THE PREFERRED EMBODIMENTS

The present invention relates to a novel soya extract which contains from about 5 to 15% by weight, preferably about 10% by weight, of sphingomyelins and from about 15 to 30% by weight, preferably above 20% by weight, of phospholipids. The sphingomyelins and the phospholipids are correspondingly present in the extract according to the invention in a ratio of from about 0.5 to 1.5 parts by weight of sphingomyelins to from about 1.5 to 3 parts by weight of phospholipids. Minor components of the extract are some sterols and long-chain fatty alcohols. In addition to the long-chain fatty alcohols and the sterols, further components of the extract are mono and diglycerides.

The invention further relates to a process for the production of the above-defined extract, which is characterized by the following stages:

a) extraction of ripe whole soya beans or oil-free soya flour using aliphatic alcohols or a mixture of these alcohols with water;

b) concentration of the extract from stage a);

c) extraction of the concentrated extract from stage b) by treatment with aliphatic hydrocarbons;

d) treatment with vegetable activated carbon and concentration of the extract from stage c) by evaporation;

e) insolubilization of the active components using aliphatic ketones;

f) filtration of the active components, and drying.

In particular, the extracts according to the invention can be produced by extracting ripe whole soya beans or oil-free soya flour using aliphatic alcohols alone or in a mixture with water, preferably using an ethanol/water mixture, such as 95% strength ethanol. Following concentration, the extract is further extracted by treatment with aliphatic hydrocarbons. Suitable aliphatic hydrocarbons for this purpose are n-hexane and n-heptane. Following treatment with vegetable activated carbon and concentration by evaporation to a reduced volume, the organic phase is dried under reduced pressure. The active components are then rendered insoluble in aliphatic ketones. An aliphatic ketone which is suitable for this purpose is, for example, acetone. Following concentration by evaporation to a reduced volume, the organic phase is dried under reduced pressure.

Preferred conditions for carrying out the individual process stages of the process according to the invention are those given below. Here and hereinafter the unit of measurement for parts by volume is 1 (litre) and that for parts by weight is kg (kilogram).

Stage a: The plant starting material is selected with regard to the desired ratio of sphingomyelins to phospholipids. For this purpose, a soya flour with a predetermined lipid content is for example used as starting material. The plant material is preferably extracted with from 12 to 17 volumes of solvent per part (by weight) of biomass. The extraction temperature is expediently above 55° C. Each extraction is expediently carried out over a period of less than 4 hours. Apart from ethanol, suitable solvents are inter alia methanol, propanol and isopropanol. These solvents can contain up to 10% of water.

Stage b: The extract is expediently concentrated at a temperature below 50° C. under reduced pressure. The extract is expediently concentrated by evaporation to an alcohol content of from65to75%.

Stage c: The concentrated extract from stage b is expediently extracted with from 0.3 to 0.6 volumes of aliphatic hydrocarbons per part (by weight) of plant material. Extraction is preferably carried out three times.

Stage d: The organic phase is treated with vegetable activated carbon and expediently evaporated to dryness at a temperature below 50° C. under reduced pressure.

Stage e: The active compounds are expediently rendered insoluble using 10 volumes of aliphatic ketones, calculated on one part (by weight) of plant material.

Stage f: The precipitate is preferably washed, for example three times; a ketone such as acetone is suitable for this purpose. Following filtration, the precipitate is expediently dried at a temperature below 50° C. under reduced pressure.

The extract according to the invention is in the form of a fat-like solid, which is readily soluble in ether and chlorinated solvents.

The content of sphingomyelins in the present extract is determined using HPLC in combination with enzymatic hydrolysis of the sphingomyelins using sphingomyelinase. The latter is an enzyme which converts the sphingomyelins into ceramides, which can be detected by HPLC or TLC.

Compared with extracts known hitherto, the extracts according to the invention are distinguished by their action on dry skin.

Table 1 shows the results obtained when dry skin was treated with the extract according to the invention, compared with a collagen treatment. The treatment according to the invention was with an emulsion, in water, containing the extract according to the invention and 2% hydroxyethylcellulose.

TABLE 1

| | Hydration (units) | | |
|---|---|---|---|
| Treatment | Starting value | After 60 min | Change % |
| Control | 43.7 ± 0.3 | 44.1 ± 0.4 | — |
| 1.5% solution of the | 44.9 ± 0.3 | 78.9 ± 0.3** | 75.7 |
| extract according to | | | |
| Example 1 | | | |
| 2% collagen (as | 42.7 ± 0.4 | 57.7 ± 0.1* | 35.6 |
| comparison) | | | |

*P < 0.05
**P < 0.001
Student t test for paired data

Skin hydration was measured as electric capacitance of the horny layer using a commercially available device (Corneometer CM 820, Courage+ Kazaka, Cologne, Germany). The mean value of five consecutive measurements was determined and recorded as the measured value. The electric capacitance is expressed by means of an arbitrary scale ranging from 0 to 100 units. Groups of 10 subjects were used.

The invention thus also extends to a pharmaceutical and cosmetic composition which contains the above extract as active component. In particular, the Invention relates to a cosmetic composition which contains this extract for the prevention or treatment of dry skin.

The products or extracts according to the invention can be formulated in a suitable manner as fluid emulsion, oil/water emulsion, water/oil emulsion, lipogel, lipstick, granular powders for the production of ready-to-use solutions or fluids or liquids which are compatible with their solubility. The concentration of the extract according to the invention introduced into the formulation is in the range 1 to 5%, preferably from 1.2 to 3.5%.

### EXAMPLES

The following example illustrates the invention.

### Example 1

### Production of Soya Extract Containing 10% by Weight of Sphingomyelins and 20% by Weight of Phospholipids

10 kg of oil-free soya flour are refluxed five times with 30 litres of 95% strength ethyl alcohol. The alcohol extracts are mixed and concentrated by evaporation under reduced pressure to 5 litres. The concentrate is extracted three times with 5 litres of n-hexane. The hexane phase is treated with vegetable activated carbon and concentrated by evaporation under reduced pressure to a reduced volume. The resulting extract is then treated with 10 volumes of acetone. The sphingomyelins and phospholipids precipitate out. Following filtration, the product is dried under reduced pressure to give 220 g of extract containing 10% by weight of sphingomyelins and 20% by weight of phospholipids.

What is claimed is:

1. A soya extract comprising one or more sphingomyelins in an amount of 5 to 15% by weight and one or more phospholipids in an amount of 15 to 30% by weight.

2. The soya extract of claim 1, wherein the amount of sphingomyelins is about 10% by weight and the amount of phospholipids is about 20% by weight.

3. A method for preparing a soya extract comprising one or more sphingomyelins in an amount of 5 to 15% by weight, and one or more phospholipids in an amount of 15 to 30% by weight, comprising:
a) extracting soya beans or oil-free soya flour with a solvent of one or more aliphatic alcohols or a mixture of one or more aliphatic alcohols and water to provide a first soya extract;
b) concentrating the first soya extract to provide a concentrated first soya extract;
c) extracting the concentrated first soya extract with one or more aliphatic hydrocarbons to provide a second soya extract;
d) treating the second soya extract with vegetable activated carbon to provide a refined second soya extract;
e.) concentrating the refined second soya extract by evaporation;
f) adding one or more aliphatic ketones to the refined second soya extract to precipitate the sphingomyelins and phospholipids;
g) separating the sphingomyelins and phospholipids from the one or more aliphatic ketones by filtration;
h) Drying the sphingomyelins and phospholipids.

4. The method of claim 3, wherein the soyabeans or oil-free soya flour is extracted with from 12 to 17 volumes of the solvent at a temperature of greater than 55° C. for a time of less than 4 hours.

5. The method of claim 3, wherein the solvent is ethanol, or an ethanol-water mixture containing up to 10% water, methanol, or a methanol-water mixture containing up to 10% water, propanol, or a propanol-water mixture containing up to 10% water, isopropanol, or an isopropanol-water mixture containing up to 10% water.

6. The method of claim 3, wherein the first soya extract is concentrated by evaporation at a temperature below 50° C. under reduced pressure to an alcohol content of from 65 to 75%.

7. The method of claim 3, wherein concentrated first soya extract is extracted with from 0.3 to 0.6 volumes of one or more aliphatic hydrocarbons per part by weight of soya beans or oil-free soya flour.

8. The method of claim 7, wherein the extraction is carried out three times.

9. The method of claim 3, wherein the refined second soya extract is concentrated by evaporation under reduced pressure and at a temperature below 50° C. to dryness.

10. The method of claim 3, wherein the one or more aliphatic ketones and the soya beans or oil-free soya flour are present in a ratio of 10 liters of aliphatic ketones per kilogram of soya beans or oil-free soya flour.

11. The method of claim 3, further comprising washing the precipitated sphingomyelins and phospholipids three times with one or more aliphatic ketones.

12. The method of claim 3, further comprising drying the precipitated sphingomyelins and phospholipids at a temperature below 50° C. under reduced pressure.

13. A soya extract according to claim 1, wherein the soya extract is in the form of a fat-like slid which is readily soluble in ether and chlorinated solvents.

14. A composition comprising the soya extract of claim 1 and one or more excipients wherein the compound of claim 1 is present in an amount of 1 to 5%.

15. A method of preventing or treating dry skin in a patient comprising administering to the patient a pharmaceutically acceptable amount of the composition of claim 14.
